(19) United States
(12) Patent Application Publication
(10) Pub. No.: US 2022/0257436 A1
(43) Pub. Date: Aug. 18, 2022

(54) My Intimates Period Pod

(71) Applicant: Merlin M. Williams, Niceville, FL (US)

(72) Inventor: Merlin M. Williams, Niceville, FL (US)

(21) Appl. No.: 17/175,619

(22) Filed: Feb. 13, 2021

(51) Int. Cl. A61F 13/551; A41D 1/22; A45C 11/00; A45C 13/10; A45C 13/00

(52) U.S. Cl. CPC A61F 13/5519; A61F 13/55135; A41D 1/22; A61F 2013/4708; A45C 13/103; A45C 13/008; A45C 11/008

(57) ABSTRACT

An article of manufacture for providing an emergency period hygiene garment is disclosed. A carrying pouch contains the components of the emergency period pod including an undergarment, a fabric cover-up, disposable sanitary wipes, and a sanitary napkin or tampon. The various components may be stored within the carrying pouch and carried by a woman for use during an unexpected start of her menstrual period. The undergarment is shown having a detachable side component with connection elements coupled to each side of the undergarment from its waist to the crotch allowing the undergarment to be separated from its front and rear portions for ease of removal.

### TECHNICAL FIELD

This application relates in general to an article of manufacture for providing a personal hygiene aid, and more specifically, to an article of manufacture for providing an emergency period hygiene garment.

### BACKGROUND

A need exists to equip menstruating women in instances related to the unplanned beginning of menarche between the ages of 9 and 15 years old. Additionally, it will be useful for emergency situation(s) for menstruating women up to the ages of perimenopause and menopause. Providing women with personal hygiene aids will positively impact their health, hygiene, and well-being by reducing anxiety and emotional stress related to a possibly unpredictable menstrual cycle and the potential of soiled clothing.

For school-aged girls, crisis mode is usually how the first-time period is dealt with, because the start of menarche varies for each individual. School-aged youth between 9-15 years old may have to rely on a school nurse to obtain emergency sanitary napkins. Also, in emergency situations resulting in soiled clothing, there is often no clean clothing immediately available at school or an office.

Therefore, a need exists for an article of manufacture for providing an emergency period hygiene garment. The present invention attempts to address the limitations and deficiencies in prior solutions according to the principles and example embodiments disclosed herein.

### SUMMARY

In accordance with the present invention, the above and other problems are solved by providing an article of manufacture for an emergency period hygiene garment according to the principles and example embodiments disclosed herein.

In one embodiment, the present invention is an article of manufacture for providing an emergency period hygiene garment. An emergency pod has carrying pouch is shown to contain the components of the emergency pod including an undergarment, the fabric cover-up, disposable sanitary wipes, and a sanitary napkin. The various components may be stored within the carrying pouch that is carried by a woman for use in a time of an unexpected beginning to her menstrual period. The undergarment is shown having a detachable side component that allows the undergarment to be worn and removed easily. A matching pair of connection elements couple to each side of the undergarment from its waist to the crotch allowing the undergarment to be separated from its front and rear portions for ease of removal.

In another aspect of the present disclosure, the emergency pod includes a waterproof carrying pouch for containing all of the items, an undergarment comprising a plurality of different material layers and a pair of detachable side components between a waistband and leg openings, one or more sanitary wipes, a sanitary napkin, and a fabric cover-up for wearing around a waist over clothing.

In another aspect of the present disclosure, the waterproof carrying pouch includes a soft neoprene case, a closing zipper, and a carabiner clip attached to the soft neoprene case.

In another aspect of the present disclosure, the carabiner clip comprises one or more retractable tools.

In another aspect of the present disclosure, the plurality of different material layers include a natural fiber inner layer, an absorbent second layer, an odor reducing third layer, and a leakproof outer layer.

In another aspect of the present disclosure, each of the pair of detachable side components between a waistband and leg openings comprises a plurality of mating snap pairs.

In another aspect of the present disclosure, each of the pair of detachable side components between a waistband and leg openings comprises one of the following: buttons, hooks, and Velcro™ attachment mechanisms.

In another aspect of the present disclosure, the one or more sanitary wipes and the sanitary napkin are contained within removable, vacuum sealed enclosure to prevent spoilage.

In another aspect of the present disclosure, the fabric cover-up and the undergarment are made of washable fabric.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIG. 1 illustrates an example woman's undergarment providing emergency period hygiene protection according to the present invention.

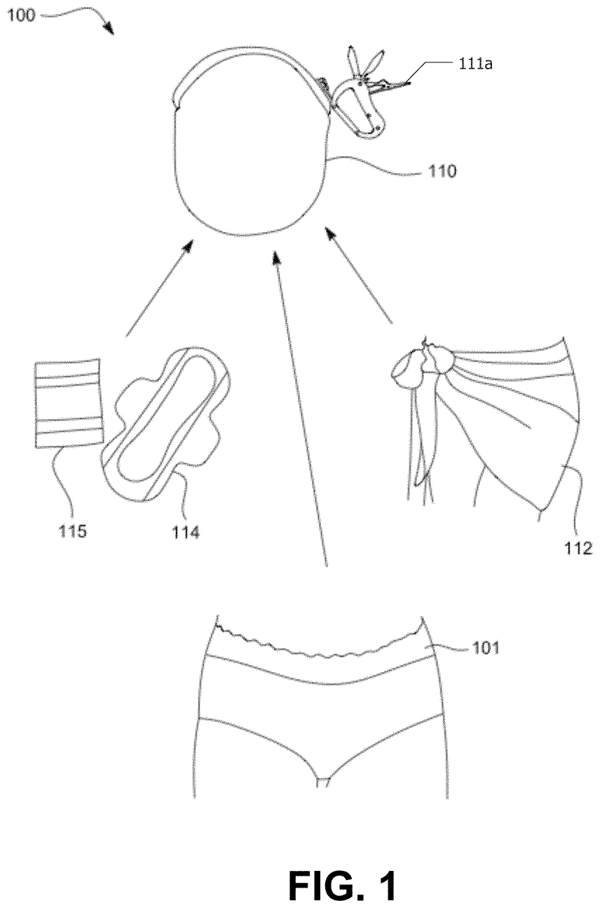

FIGS. 2a-c illustrate an example embodiment of the components that are part of an article of manufacture for an emergency period hygiene garment according to the present invention.

FIG. 3a illustrates multiple fabric layers within an article of manufacture for an emergency period hygiene garment insert according to the present invention.

FIG. 4 illustrates a side connection that is part of an example woman's undergarment providing emergency period hygiene protection according to the present invention.

### DETAILED DESCRIPTION

This application relates in general to an article of manufacture for providing a personal hygiene aid, and more specifically, to an article of manufacture for providing an emergency period hygiene garment according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “woman,” and “user” refer to an entity, e.g., a human, using an emergency period hygiene garment associated with the invention. The term user herein refers to one or more users.

The term “invention” or “present invention” refers to the invention being applied for via the patent application with the title “My Intimates Period Pod.” Invention may be used interchangeably with undergarment.

In general, the present disclosure relates to an article of manufacture for providing an emergency period hygiene garment. To better understand the present invention, FIG. I illustrates an example woman's undergarment providing emergency period hygiene protection according to the present invention. A carrying pouch 110 is shown to contain the components of the emergency pod 100 including an undergarment 101, the fabric cover-up 112, disposable sanitary wipes 115, and a sanitary napkin 114. The various components 101, 112-114 may be stored within the carrying pouch 110 that is carried by a woman for use when her menstrual period unexpectedly begins. The entire emergency pod I 00 contains biodegradable feminine hygiene wipe(s) and a sanitary napkin with wings that is made of a material of organic cotton or a tampon in an adult version of the pod 100. The sanitary wipes 115 Feminine wipes have a basic size is typically is 14×17 cm that may also be fragrance-free and hypoallergenic. Items within the emergency pod I 00 may each be enclosed within a removable, vacuum sealed enclosure to prevent spoilage.

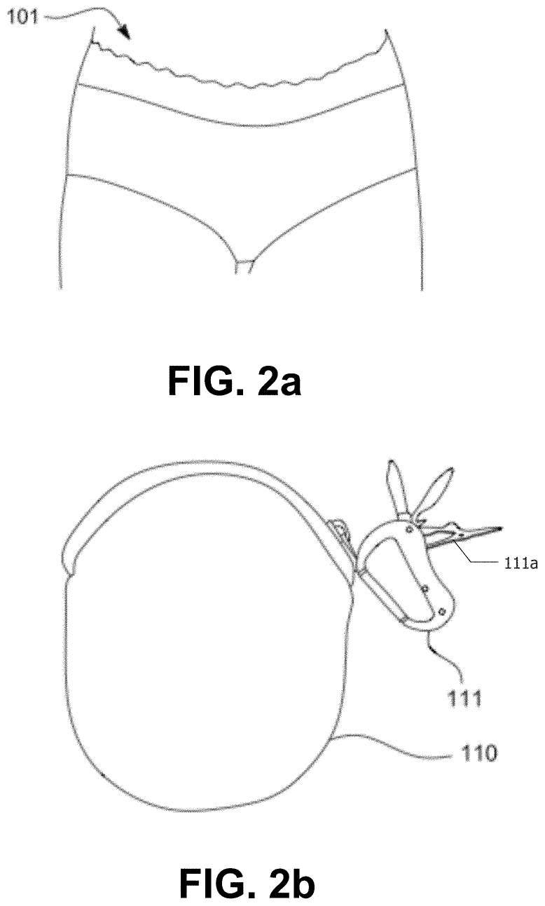

The emergency pod 100 also includes a polyester blend fabric cover-up to camouflage soiled clothing. Each of these items may be enclosed within a sealed and vacuum-sealed package within in a reusable, soft neoprene, waterproof pouch 110. The entire emergency pod I 00 is portable and discretely packaged in the reusable, soft neoprene, waterproof carrying pouch 110 with a carabiner clip to easily attach to bag or clothing. The carabiner clip 111 may include one or more retractable tools 11la including a small knife, scissors, a nail file and the like. The fabric cover-up and the undergarment are made of washable fabric. Each of these items is described in additional detail below in reference to FIG. 2.

FIGS. 2a-c illustrate an example embodiment of the components that are part of an article of manufacture for an emergency period hygiene garment according to the present invention. FIG. 2a shows the undergarment 101 as worn by a user to provide protection during an unexpected period of menstrual bleeding. The undergarment IOI is a feminine undergarment, detachable on both sides, that is leak-proof and multi-layered polyester and cotton-blend fabrics. The undergarment 101 may be worn as any other woman's undergarment. The undergarment 101 may be made in different sizes to accommodate various different women.

FIG. 2b shows the carrying pouch 110 that is made to discretely contain and carry the items of the emergency pod 100. The carrying pouch 110 is a reusable, soft neoprene, waterproof pouch 110 with a carabiner clip 111 to easily attach to a bag or clothing. The carrying pouch 110 may include a zipper or similar closing mechanism to keep the contents of the emergency pod I 00 secure and out of sight. The carabiner clip 111 may include other useful items that a woman may carry if desired. The carabiner clip 111 may be coupled to a zipper pull for convenience. In a preferred embodiment, the carabiner clip 111 includes one or more retractable tools 111a such as a pair of scissors 111a.

FIG. 2c shows the fabric cover-up 112 as it may be worn by a woman. When these unexpected events occur, a user's clothing may become soiled and provide a source of embarrassment as she manages to continue the daily activities until a change of clothing may be obtained and used to replace the soiled outfit. The fabric cover-up 112 comprises a fashionable piece of fabric that's worn around the woman's waist is any desirable arrangement until other clothing becomes available. The fabric cover-up 112 may be made of many different types of fabric having patterns, colors, and designs that provide the woman an attractive appearance when worn. In one embodiment, the fabric cover-up 112 is a Waist Cover-up (Pareo), size 50 cm, made from a material including spandex, polyester, and chiffon-blend.

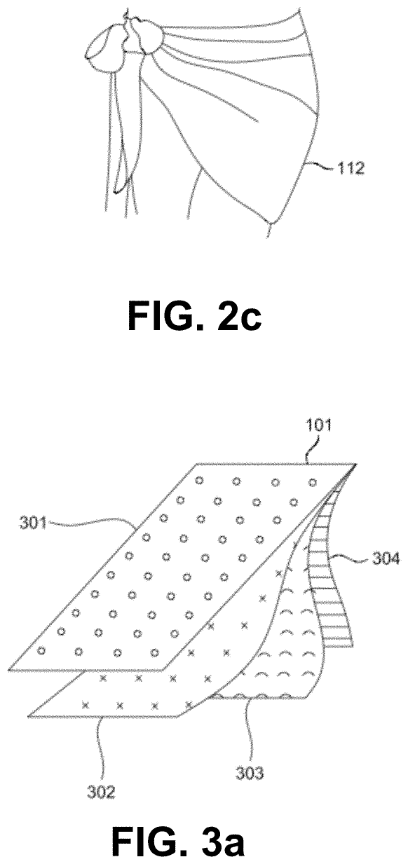

FIG. 3 illustrates multiple fabric layers within an article of manufacture for an emergency period hygiene garment insert according to the present invention. FIG. 3a shows the several layers of different materials that comprise a leak-proof and multi-layered undergarment 101 made from polyester and cotton-blend fabrics. The garment layers include a natural fiber material layer 301 to come in contact with the user's skin, an absorbent fabric layer 302 to absorb and contain bodily fluids, an odor removing layer 303 to inhibit odor-causing bacteria, and a leakproof outer layer 304 to continue to prevent additional soiling of clothing. The natural fiber layer 301 wicks any fluids into the inner layers to provide a dry feeling upon the user's skin. The absorbent layer 302 accepts the fluids from the natural fiber layer 301 for suspension within its materials. Absorbent, moisture wicking layer(s) 302-303 traps moisture and reduce odor. The outer layer 304 is leakproof to prevent any materials from within the fabrics to reach the user's clothing and thus become visible.

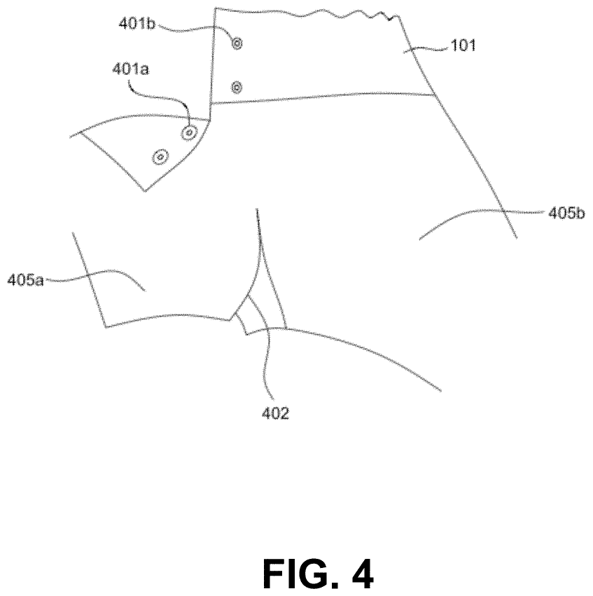

FIG. 4 illustrates a side connection that is part of a woman's undergarment providing emergency period hygiene protection according to the present invention. The undergarment 101 is shown having a detachable side component 402 between a waistband and leg openings that allows the undergarment 101 to be worn and removed easily. A matching pair of connection elements 401a-b couple each side of the undergarment 101 from its waist to the crotch allowing the undergarment 101 to be separated from its front and rear portions 405a-b. The matching connection elements 401a-b are shown as a series of mating snaps in the example embodiment of FIG. 4. Other connection elements include buttons, hooks, Velcro,™ and related mechanisms. When the undergarment 101 has been used, the woman may disconnect the two portions 405a-b from each other to remove the undergarment 101 and then roll the undergarment 101 around itself to allow only the outer leakproof layer 304 to be seen before washing of the undergarment.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture for providing an emergency period hygiene garment, the article comprises:
a waterproof carrying pouch for containing all of the items;
an undergarment comprising a plurality of different material layers and a pair of detachable side components between a waistband and leg openings;
one or more sanitary wipes;
a sanitary napkin; and
a fabric cover-up for wearing around a waist over clothing.

2. The article of manufacture according to claim 1, wherein the waterproof carrying pouch comprises:
a soft neoprene case;
a closing zipper; and
a carabiner clip attached to the soft neoprene case.

3. The article of manufacture according to claim 2, wherein the carabiner clip comprises one or more retractable tools.

4. The article of manufacture according to claim 1, wherein the plurality of different material layers comprise:
a natural fiber inner layer;
an absorbent second layer;
an odor reducing third layer; and
a leakproof outer layer.

5. The article of manufacture according to claim 4, wherein each of the pair of detachable side components between a waistband and leg openings comprises a plurality of mating snap pairs.

6. The article of manufacture according to claim 4, wherein each of the pair of detachable side components between a waistband and leg openings comprises one of the following: buttons, hooks, and Velcro™ attachment mechanisms.

7. The article of manufacture according to claim 4, wherein the one or more sanitary wipes and the sanitary napkin are contained within removable, vacuum sealed enclosure to prevent spoilage.

8. The article of manufacture according to claim 1, wherein the fabric cover-up and the undergarment are made of washable fabric.
